(19) United States
(12) Patent Application Publication
(10) Pub. No.: US 2017/0056441 A1
(43) Pub. Date: Mar. 2, 2017

(54) NUTRITIONAL SUPPLEMENTS FOR THE PREVENTION AND RELIEF OF MUSCLE CRAMPS AND RELATED METHODS

(71) Applicants: Ademiloluwa A. ADENIJI, Fresno, CA (US); Beni ADENIJI, Fresno, CA (US)

(72) Inventors: Ademiloluwa A. ADENIJI, Fresno, CA (US); Beni ADENIJI, Fresno, CA (US)

(21) Appl. No.: 15/247,745

(22) Filed: Aug. 25, 2016

Related U.S. Application Data
(60) Provisional application No. 62/209,804, filed on Aug. 25, 2015.

(51) Int. Cl. A61K 33/06; A61K 33/00; A61K 31/197; A61K 31/593

(52) U.S. Cl. CPC A61K 33/06; A61K 31/593; A61K 33/00; A61K 31/197

(57) ABSTRACT

Nutritional supplements that reduce the occurrence of and/or prevent and relieve exercise-associated muscle cramps (EAMC) as well as other forms of skeletal muscle cramping are disclosed. The supplements typically comprise Vitamin D3, one or more potassium compounds, creatine monohydrate and magnesium glycinate (and/or other magnesium compounds), and advantageously reduce or eliminate the acute pain, disruptions in the ability to compete, and the associated frustration and disappointment of EAMC and other muscle cramps Aspects of the present invention also provide methods of formulating and using such nutritional supplements for the prevention and relief of muscle cramping.

### CROSS-REFERENCE TO RELATED APPLICATIONS

This application claims the benefit of U.S. Provisional Patent Application No. 62/209,804 filed Aug. 25, 2015. The text and contents of that provisional patent application are hereby incorporated into this application by reference as if fully set forth herein.

### FIELD OF THE INVENTION

The present invention generally relates to the field of nutritional supplements. Specifically, embodiments of the present invention relate to nutritional supplements formulated and administered for the prevention and relief of muscle cramps.

### DISCUSSION OF THE BACKGROUND

Muscle cramps, a sudden and involuntary contraction of one or more muscles, can cause excruciating pain and make it temporarily impossible to use the affected muscle. Such cramps may be caused by long periods of exercise or physical labor, particularly in hot weather, or may be caused by certain medical conditions or medications.

Skeletal muscle cramps that occur during or shortly following exercise in healthy individuals with no underlying metabolic, neurological, or endocrine pathology have been termed “exercise-associated muscle cramps” (EAMC). EAMC may be recognized by acute pain, stiffness, visible bulging or knotting of the muscle, and possible soreness that can last for a varying duration of time (from a few seconds to several days) after the initial cramping event. EAMC is common in both recreational and competitive athletes. The cause of EAMC continues to be unresolved, although some health care professionals believe it to be caused by a decreased supply of oxygen, lactic acid build up, dehydration and/or electrolyte imbalance. Other health care professionals believe the cause to be neuromuscular, in other words, caused by muscle overload and neuromuscular fatigue. Yet others believe that muscle cramps are caused by the nerve—when motor neurons in the spinal cord fire spontaneously and repetitively. In any case, the electrolyte imbalance theory of causation for muscle cramping is not the sole mechanism, but appears to play a role in muscle cramping in situations of excessive heat or dehydration.

The result of the occurrence of EAMC is that professional and amateur athletes have been removed from competition and game play. The inability of athletes to continue in competitions produces disruptions, frustration and disappointment. For a swimmer, the consequences can be even more dire. Although there have been extensive medical, nutritional and dietary studies conducted and published, previously there has been no consistent, reliable solution to EAMC.

Regardless of the cause, current treatments and prevention strategies for EAMC such as ingesting fluids containing electrolytes, stretching and other treatment and prevention strategies have been largely unsuccessful. As a result, it is desirable to provide nutritional supplement formulations and methods for the effective prevention and rapid relief of EAMC and other types of muscle cramping.

### SUMMARY OF THE INVENTION

Embodiments of the present invention advantageously provide nutritional supplements and methods for formulating and administering such nutritional supplements for the prevention and relief of EAMC and other forms of muscle cramping. These supplements are unique mixtures most typically comprising cholecalciferol (as Vitamin D3), magnesium glycinate, one or more potassium salts (e.g., potassium chloride, potassium bicarbonate and/or potassium citrate) and/or creatine monohydrate. The supplements and methods disclosed herein rapidly provide nutritional elements and electrolytes at the neuromuscular junction in order to facilitate efficient muscle energy production (adenosine triphosphate (ATP), nutrient uptake and tendon relaxation via the Golgi tendon organ (GTO) inhibitory activity.

Also disclosed are methods for formulating the nutritional supplements, typically comprising weighing the appropriate quantities of base compounds, pouring the compounds into a resealable bag or a mixing machine, shaking the bag and/or appropriately mixing the base compounds until they are thoroughly blended, breaking up any large particles, if necessary, and re-shaking or re-mixing. In some embodiments, the method may also comprise pouring the mixture into a capsule filling machine, and encapsulating the formulation in an appropriate size capsule.

Further disclosed are methods of storing and using the nutritional supplements comprising, for an athlete under 200 lbs., taking two (2) capsules at least fifteen (15) minutes before exercise with about 300-500 ml (12-16 fl. oz.) of water or a sports drink. For persons over 200 lbs., three (3) capsules should be taken at least fifteen minutes before exercise. In some instances, the method may also include taking an additional two capsules (or three capsules if over 200 lbs) for high intensity sustained exercise such as marathon running or soccer every two (2) to three (3) hours during continued exercise.

In very hot or high humidity playing conditions the first repeat dose should taken after 45-60 minutes then 2-3 hourly thereafter. However, the maximum number of doses which may be taken in a 24-hour period is twelve capsules.

Embodiments of the present invention advantageously provide nutritional supplements for preventing and rapidly relieving muscle cramps by addressing the multiple mechanisms of muscle cramp generation, including neuromuscular junction activity (especially stimulating the Golgi tendon organ inhibitory action) and electrolyte repletion, which are thought to be the cause of muscle cramping, including cramping during exercise.

It is to be understood that both the foregoing general description and the following detailed description are exemplary, but not restrictive, of the invention. A more complete understanding of the embodiments of the nutritional supplements and methods disclosed herein will be afforded to those skilled in the art.

### BRIEF DESCRIPTION OF THE DRAWINGS

FIG. 1 shows the chemical structure of cholecalciferol, a compound used in embodiments of the supplements of the present invention.

FIG. 2 shows the chemical structure of magnesium glycinate, a compound used in embodiments of the supplements of the present invention.

FIG. 3 shows the chemical structure of potassium chloride, a compound used in embodiments of the supplements of the present invention.

FIG. 4 shows the chemical structure of potassium bicarbonate, a compound used in embodiments of the supplements of the present invention.

FIG. 5 shows the chemical structure of creatine monohydrate, a compound used in embodiments of the supplements of the present invention.

FIG. 6 shows the chemical structure of potassium citrate, a compound used in embodiments of the supplements of the present invention.

### DETAILED DESCRIPTION OF THE PREFERRED EMBODIMENTS

Reference will now be made in detail to the preferred embodiments of the invention. While the invention will be described in conjunction with the preferred embodiments, it will be understood that they are not intended to limit the invention to these embodiments. On the contrary, the invention is intended to cover alternatives, modifications, and equivalents that may be included within the spirit and scope of the invention. Furthermore, in the following detailed description of the present invention, numerous specific details are set forth in order to provide a thorough understanding of the present invention. However, it will readily be apparent to one skilled in the art that the present invention may be practiced without these specific details.

Embodiments of the present invention advantageously provide nutritional supplement formulations and methods for preventing and rapidly relieving exercise-associated (and other) muscle cramps. Painful cramps occur when a muscle contracts while in a shortened state. These typically occur in a single group of large muscles that span 2 joints such as the hamstrings, quadriceps, and triceps, but are also seen in the small muscles subjected to repetitive excessive use, such as the calf muscles, and hands, as with writer's cramp.

It is well known that muscle cramps occur in differing environmental conditions (e.g. hot ambient temperatures, cold temperatures, humid conditions [e.g. such as those to which miners are exposed]), but cramping may also occur in dry conditions (e.g., writer's cramping in dry indoor environments), and in diverse populations. Therefore, it is unlikely that a single factor is causal in all instances.

Embodiments of the present invention are novel and unobvious combinations of substances which act on the neuromuscular junction and increase adenosine triphosphate (ATP) uptake and utilization, which is the primary source of energy in all living cells. Although the subject specification refers to exercise-associated muscle cramps (EAMC), the formulations and methods described herein may also be effective for the relief of cramping unrelated to active exercise.

The nutritional supplements of the present invention rapidly provide the critical elements and electrolytes at the neuromuscular junction to facilitate efficient muscle energy uptake and muscle relaxation via the Golgi tendon organ inhibitory activity.

The basic components of the formulations typically comprise (i) cholecalciferol (as Vitamin D3), (ii) magnesium glycinate, (iii) one or more potassium compounds/salts (e.g., potassium chloride, potassium bicarbonate, potassium citrate, potassium malate, potassium glutamate, etc.) and/or (iv) creatine monohydrate. Preferably, all of these components are used in the formulation as they contribute to the formulation's uniquely efficacious activity in reducing occurrence of cramping during muscle activity. However, in some embodiments, less than all of the components may be used.

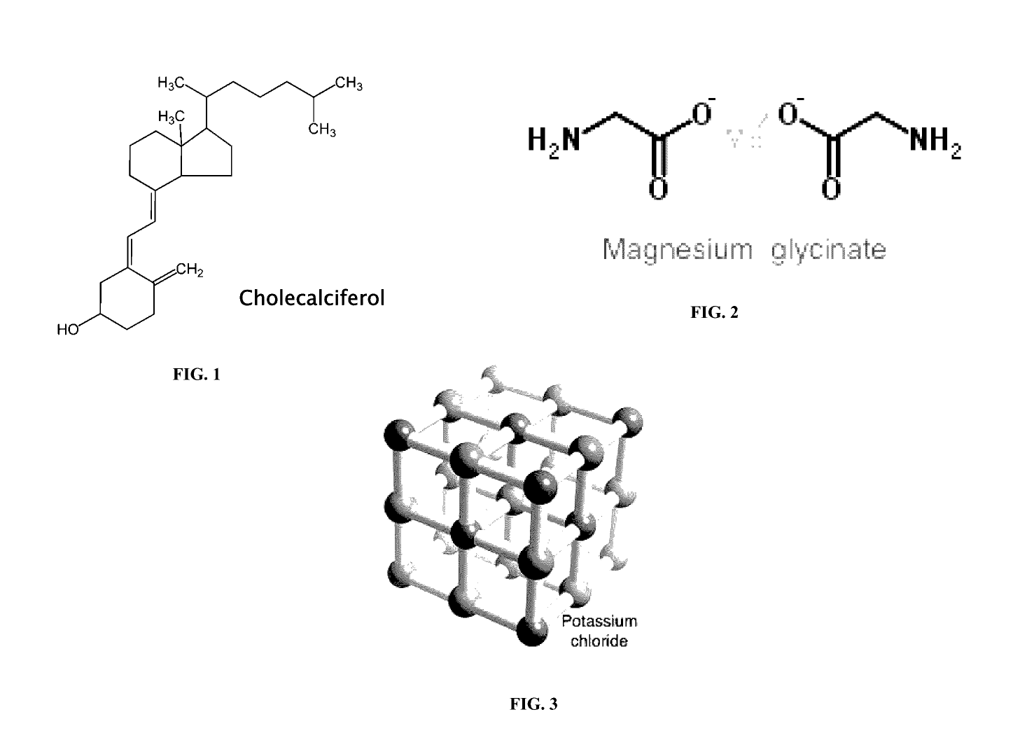

Referring now to FIG. 1, therein is shown the chemical structure of cholecalciferol. Cholecalciferol (Vitamin D3) receptors have been demonstrated in muscle fibers and the neuromuscular junction. Deficiency of Vitamin D is associated with muscle weakness, increased frequency of falls, and with muscle cramps. Ingested Vitamin D is transferred from the circulation and body fluids into tissues and the bone for storage from where slow release occurs over time to meet the body's basal needs. With rampant Vitamin D insufficiency demonstrated in many populations studied, and the significant increase in demand during vigorous exercise, functioning of the Golgi tendon organs (GTOs) is often hampered.

GTOs are responsible for inhibitory impulses to the muscle spindle which produces muscle relaxation after contraction. Providing a readily available source of cholecalciferol immediately prior to exercise maintains blood levels (prior to transfer to storage forms) and helps maintain the balance between excitatory impulses from muscle spindles and the critical inhibitory impulses from GTOs, ensuring normal contraction-relaxation signaling of the alpha motor neuron. The unique formulations of the present invention ensure rapid uptake of recently ingested cholecalciferol before distribution into storage forms, producing enhanced muscle and nerve calcium coupling and signal transduction.

Referring now to FIG. 2, therein is shown the chemical structure of magnesium glycinate. Magnesium glycinate is a combination of magnesium, an essential mineral and glycine, a non-essential amino acid. It is easily absorbed by the body, and provides a high level of bioavailability. Magnesium deficiency has been described as a major cause of muscle cramps. The immediate availability of magnesium glycinate in delivering this essential cation (carries a net positive electrical charge), to the exercising muscle end plate and ATP-ase pump is an important component of the activity of embodiments of the present invention. Although magnesium glycinate is typically used in preferred embodiments, one or more other forms of magnesium (e.g., magnesium L-threonate, magnesium taurate, magnesium chloride, magnesium lactate, magnesium carbonate, magnesium citrate, etc.) may be used in some embodiments or may be used in combination with magnesium glycinate.

Embodiments of the present invention may also utilize one or more potassium compounds. For example, in preferred embodiments, one or more of potassium chloride, potassium citrate and/or potassium bicarbonate are utilized. However, other embodiments may also comprise potassium gluconate, potassium sulfate, potassium malate and/or potassium bitartrate.

Potassium is an important element, the lack thereof which is implicated in electrolyte imbalance culminating in muscle cramps. Potassium is involved in every cellular function and is a critical component of nerve and muscle activity and ATP utilization. The “recommended daily allowance” suggested for potassium is high and very difficult to attain with the modern American diet. The Food and Nutrition Board of the Institute of Medicine suggests an adequate intake of potassium is about 4.7 grams/day and the FDA suggest an intake of 3.6 grams/day. The typical diet contains less than 2 grams/day.

Most of the body's potassium is stored inside the cells and transported in small amounts to the extracellular compartment by carrier molecules in the cell wall. Potassium and sodium exchange is a dynamic process which is coupled with ATP production. A pre-exercise potassium, cholecalciferol and creatine monohydrate combination optimizes extracellular potassium availability at the neuromuscular spindle to meet the increased demand during vigorous exercise.

At least 20 milliequivalents (mEq) of potassium is generally recommended for athletes weighing up to 200 lbs. An additional 10 mEq should be added for 200-300 lb. body weight (hence 30 mEq).

Potassium supplementation at these levels raises no safety concerns in individuals that are healthy enough for sporting activity. Individuals with kidney failure and heart disease must be cleared by their health provider before using the nutritional supplements of the instant invention, or engaging in vigorous sporting activity. The instant nutritional supplements should not be used for any other purpose aside from pre-exercise enhancement of muscle energy production and cramp prevention.

In FIG. 3 is shown the chemical structure of potassium chloride. Potassium chloride is a metal halide salt composed of potassium and chloride. It is odorless and has a white or colorless vitreous crystal appearance. Potassium chloride dissolves easily in water and has a salty taste when so dissolved. Potassium chloride is used in the treatment of hypokalemia (low concentration of potassium in the blood) and as an electrolyte replenishment.

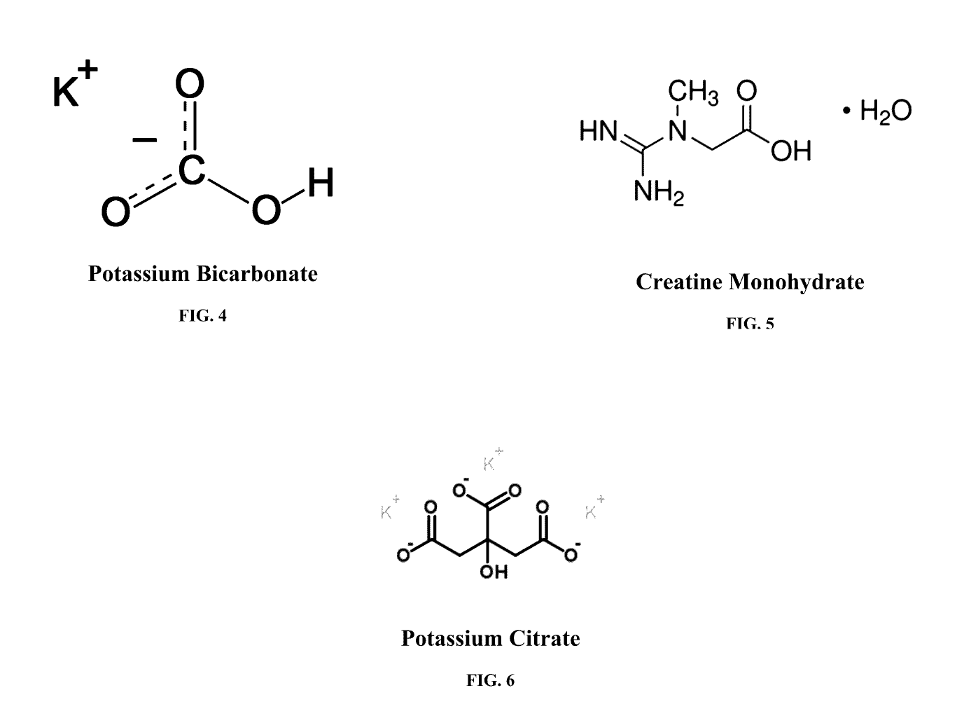

Referring now to FIG. 4, therein is shown the chemical structure of potassium bicarbonate. Potassium bicarbonate is a colorless, odorless, slightly basic, salty substance. It occurs as a soft white granular powder and it is very rarely found in its natural form as a mineral called kalicinite. This is used in some East African tribes as a native salt for cooking, and it may be an environmental factor that contributes to the superior endurance of African long distance runners. Potassium bicarbonate is an important physiologic buffer of organic acids in the body and may be important in reducing the effects of lactic acid generation from anerobic respiration in actively working muscles.

The U.S. Food and Drug Administration (FDA) recognizes potassium bicarbonate as safe. There is no evidence of human carcinogenicity, no adverse effects of overexposure and it is often added to bottled water for taste enhancement. There are three (3) oxygen atoms in each molecule of potassium bicarbonate and these are readily released with hydrolysis in the stomach.

FIG. 5 shows the chemical structure of creatine monohydrate. Creatine (as creatine monohydrate) enhances muscle phosphocreatine and increases efficiency of energy metabolism in the form of ATP (adenosine triphosphate) production and utilization. In small amounts, the weight gain, cramping and diarrhea associated with the high dose creatine is avoided (usual maintenance dosing for body building is about 2 gm per day of creatine). The nutritional supplements disclosed herein are not designed for building muscle mass. The low dose creatine enhances uptake and delivery of cholecalciferol, potassium and magnesium by Golgi tendon organs because creatine is actively utilized for ATP production in vigorously exercising muscles.

Referring now to FIG. 6, therein is shown the chemical structure of potassium citrate. Potassium citrate appears as a white, odorless, crystalline powder that is water soluble, and is rapidly absorbed when given by mouth. It is an alkaline salt that is often used to reduce the pain and frequency of urination caused by high acidic urine and to treat kidney stones. It is also an important physiologic buffer for organic acids in the body.

Embodiments of the nutritional supplements disclosed enhance performance, particularly for high intensity activity of relatively short duration (2-3 hours). Repeat dosing is recommended after 1-3 hours of intense activity in order to maintain efficiency of Golgi tendon organ inhibitory cycling.

The nutritional supplements of the present invention may be formulated as a mixture comprising up to 5.0% cholecalciferol (as Vitamin D3), from 7.5% to 35.0% magnesium glycinate (or other magnesium compounds; e.g., magnesium L-threonate, magnesium taurate, magnesium chloride, magnesium lactate, magnesium carbonate, magnesium citrate, etc.), from 60% to 85.0% potassium salts (e.g., potassium chloride, potassium bicarbonate, potassium citrate, potassium malate, potassium glutamate, etc. providing from 25.0% to 35.0% elemental potassium), and from 7.5% to 25% creatine monohydrate. Percentages shown are by weight compared to the total weight of the formation.

Most preferably, the nutritional supplements are formulated as a dry mixture comprising up to 1.0% cholecalciferol (as Vitamin D3), from 9.0% to 10.0% magnesium glycinate, from 70.0% to 82.0% potassium compounds/salts (providing from 25.0% to 30.0% elemental potassium) and from 9.0% to 21.0% creatine monohydrate. Percentages shown are by weight compared to the total weight of the formation.

It is expressly intended that all ranges broadly recited in this document include all narrower ranges that fall within the broader ranges.

In some aspects, the potassium compounds in the nutritional supplement mixtures of the present invention comprise one or more of potassium citrate, potassium bicarbonate and potassium chloride. However, other potassium compounds may also be used in the formulation (e.g., potassium chloride, potassium bicarbonate, potassium citrate, potassium malate, potassium glutamate, etc.) In typical embodiments, potassium citrate, potassium bicarbonate and potassium chloride are used in the formulation, the combination providing at least 25% elemental potassium when compared to the total weight of the nutritional supplement mixtures. Most preferably, elemental potassium comprises between 29.0% and 30% of the total weight of the nutritional supplement mixtures. The potassium compounds are most typically used in their granular form.

The weight of Vitamin D3 is calculated based on the use of Vitamin D3 powder having 102,900 International Units (IU) per gram. However, the IU per gram may vary for different lots of the D3 powder and for different manufacturers. Consequently, the weight of Vitamin D3 in the formulation may be adjusted accordingly.

The weight of magnesium glycinate is based on 15% weight of elemental magnesium to weight of magnesium glycinate (w/w). However, if the weight of elemental magnesium in the magnesium glycinate compound varies or one or more other magnesium compounds are utilized (e.g. magnesium L-threonate, magnesium taurate, magnesium chloride, magnesium lactate, magnesium carbonate, magnesium citrate, etc.) having a different w/w, then the formulation may be adjusted accordingly.

Creatine monohydrate is typically used in the formulation in a powder form.

### Exemplary Formulations

### Per 100 Capsules

The following examples of particular embodiments are given for illustrative purposes only. The examples are not intended to be a limitation on the scope or practice of the invention. Numerous variations of the present invention are possible without departing from the spirit and scope of the invention. The following examples are given on a per 100 capsule basis and are prepared by a simple mixing procedure.

### Example 1

Vitamin D3 powder (102,900 IU/gm): 0.750 gm; magnesium glycinate (15% w/w): 9.500 gm; potassium chloride (USP granular): 4.500 gm; potassium bicarbonate (USP granular): 80 gm; and creatine monohydrate powder: 25.700 gm.

### Example 2

Vitamin D3 powder (102,900 IU/gm): 1.151 gm; magnesium glycinate (15% w/w): 12.500 gm; potassium chloride (USP granular): 6.300 gm; potassium bicarbonate (USP granular): 75.500 gm; and creatine monohydrate powder: 15.000 gm.

### Example 3

Vitamin D3 powder (102,900 IU/gm): 0.972 gm; magnesium glycinate (15% w/w): 10.000 gm; potassium chloride (USP granular): 5.000 gm; potassium bicarbonate (USP granular): 3.000 gm; potassium citrate (USP granular): 76.500 gm; and creatine monohydrate powder: 12.500 gm.

### Example 4

Vitamin D3 powder (102,900 IU/gm): 1.250 gm; magnesium glycinate (15% w/w): 11.900 gm; potassium chloride (USP granular): 4.800 gm; potassium bicarbonate (USP granular): 38.000 gm; potassium citrate (USP granular) 42.500 gm and creatine monohydrate powder: 22.730 gm.

### Example 5

Vitamin D3 powder (102,900 IU/gm): 1.000 gm; magnesium glycinate (15% w/w): 10.000 gm; potassium chloride (USP granular): 5.00 gm; potassium citrate (USP granular): 80 gm; and creatine monohydrate powder: 25.00 gm.

### Example 6

Vitamin D3 powder (102,900 IU/gm): 0.972 gm; magnesium glycinate (15% w/w): 10.000 gm; potassium citrate (USP granular): 80.000 gm; potassium bicarbonate (USP granular): 2.500 gm; potassium chloride (USP granular): 2.500 gm; creatine monohydrate powder: 10.000 gm.

### Example 7

Vitamin D3 powder (102,900 IU/gm): 1.000 gm; magnesium glycinate (15% w/w): 15.00 gm; potassium citrate (USP granular): 75 gm; potassium bicarbonate (SP granular): 5.00 gm; creatine monohydrate powder 15.000 gm.

As indicated above, these are just a few examples of the compositions of the nutritional supplements of the present invention and are for illustrative purposes only.

### Exemplary Methods of Formulating

Equipment and devices that may be utilized to prepare the formulation comprise an analytical or precision laboratory balance, (e.g., Ohaus™ Explorer balance, Adam Equipment™ Nimbus, Mettler Toledo™ Excellence, etc.), weigh boats, one or more mixing containers such as reclosable bags (e.g., Zip Loc™ or similar) or mixing machine (e.g., Ross™ Pharmaceutical Mixer, Design Integrated Technology (DIT)™, etc.), a capsule filling machine and vibrator (e.g., Jaansum™, Torpac™ or similar), acrylic roller (or other appropriate device for breaking up large particles), if necessary, and 20 dram amber vials (or other appropriate vials). In some embodiments (e.g., when the supplements are not encapsulated), a capsule filling machine and vibrator may not be necessary.

Methods for formulating the nutritional supplements of the present invention comprise: (1) weighing the appropriate quantities of cholecalciferol (as Vitamin D3), magnesium glycinate (and/or one or more other magnesium compounds), potassium compounds/salts (e.g. potassium chloride, potassium bicarbonate, potassium citrate, potassium malate, potassium glutamate, etc.) and/or creatine monohydrate; (2) pouring the dry compounds into a mixing container (e.g. reclosable bag) or mixing machine; and (3) mixing and/or shaking the compounds (in some embodiments, continuously) until all compounds are thoroughly blended. In some embodiments, the method may also comprise: (4) breaking up any large particles using an acrylic roller or similar device; and (5) re-mixing. In some further embodiments, the method may also comprise: (6) pouring and/or otherwise transferring the contents into a capsule machine; and (7) encapsulating in the appropriate size capsule. For example, capsule #00 clear with a clear capsule cap or other suitable capsule may be utilized. The compounds may be mixed and added in any order, and in some embodiments, steps of the method (e.g., breaking up large particles by rolling an acrylic roller or other device) may be performed on the individual compounds before they are weighed and added. The filled capsules should be stored in an air-tight container and protected from light and/or heat.

Most typically the nutritional supplements of the present invention are encapsulated, wherein each capsule comprises approximately 1000 IU of Vitamin D3 (approximately 9.72 mg), 850 mg of one or more potassium compounds (most typically a combination of potassium citrate, potassium bicarbonate and potassium chloride providing at least 300 mg of elemental potassium), 100 mg of creatine monohydrate and 100 mg of magnesium glycinate. However, in other embodiments, other quantities of the compounds may be formulated as described above and may be encapsulated. When the formulation is not encapsulated, a dose typically comprises approximately 1050 mg to 1250 mg of the mixture depending on the exact formulation utilized.

### Exemplary Methods of Using

Typically, the nutritional supplements of the present invention should be used within one year of filling. For best results, the nutritional supplements should be taken at least fifteen (15) minutes before exercise with about 300-500 ml (12-16 fl. oz.) of a hypnotic solution such as water or a sports drink. Two (2) capsules (or the equivalent) are recommended for athletes weighing less than 200 lbs. For weight between 200 and 300 lbs., 3 capsules (or the equivalent) are recommended. If high intensity activity is undertaken for long periods of time, or in high humidity and heat, the dose may be repeated one (1) to two (2) hours after the initial dose up to the maximum of twelve capsules in a 24-hour period. Cramping in children less than 12 years of age is uncommon in the absence of medical illness, and thus, parents of children under 12 should consult a physician before use.

What is claimed is:

1. A nutritional supplement for preventing and relieving muscle cramps comprising a mixture of:
Vitamin D3;
one or more potassium compounds;
creatine monohydrate; and
magnesium glycinate.

2. The nutritional supplement of claim 1, wherein the one or more potassium compounds comprise one or more of potassium citrate, potassium bicarbonate and potassium chloride.

3. The nutritional supplement of claim 1, wherein the one or more potassium compounds comprise potassium citrate, potassium bicarbonate and potassium chloride.

4. The nutritional supplement of claim 1, wherein the one or more potassium compounds comprise at least 70% by weight, of a total weight of the nutritional supplement.

5. The nutritional supplement of claim 1, wherein magnesium glycinate comprises at least 9.0% by weight, of a total weight of the nutritional supplement.

6. The nutritional supplement of claim 1, wherein the weight of creatine monohydrate comprises at least 9.0% by weight, of a total weight of the nutritional supplement.

7. The nutritional supplement of claim 1, wherein the weight of Vitamin D3 comprises up to 1% by weight, of a total weight of the nutritional supplement.

8. The nutritional supplement of claim 1, wherein the supplement is encapsulated, and the mixture contained in each capsule comprises:
1000 IU of the Vitamin D3;
850 mg of the one or more potassium compounds;
100 mg of the creatine monohydrate; and
100 mg of the magnesium glycinate.

9. A method for formulating the nutritional supplement of claim 1, the method comprising:
weighing amounts of Vitamin D3, each of one or more potassium compounds, creatine monohydrate, and magnesium glycinate;
pouring the weighed amounts into a mixing container;
mixing and/or shaking the amounts until blended.

10. The method of claim 9, further comprising encapsulating the blended mixture into capsules.

11. The method of claim 10, wherein encapsulating the blended mixture comprises using a capsule filling machine.

12. The method of claim 9, further comprising storing the capsules in an air tight container.

13. The method of claim 9, further comprising protecting the capsules from light.

14. The method of claim 9, wherein the weighed amounts comprise:
up to 1.0% by weight of Vitamin D3;
70.0% to 82.0% by weight of potassium compounds;
9.0% to 10.0% by weight of magnesium glycinate; and
9.0% to 21.0% by weight of creatine monohydrate;

15. A method of preventing and relieving muscle cramps, the method comprising, administering an effective amount of a nutritional supplement, the nutritional supplement consisting essentially of:
up to 1.0% by weight of Vitamin D3;
70.0% to 82.0% by weight of potassium compounds;
9.0% to 10.0% by weight of magnesium glycinate; and
9.0% to 21.0% by weight of creatine monohydrate;
based on a total weight of the supplement.

16. The nutritional supplement of claim 14, consisting essentially of between 9% and 10% by weight of magnesium glycinate, based on a total weight of the nutritional supplement.

17. The nutritional supplement of claim 14, consisting essentially of between 9% and 10% of creatine monohydrate, based on a total weight of the nutritional supplement.

18. The nutritional supplement of claim 14, consisting essentially of between 20% and 21% creatine monohydrate, based on a total weight of the nutritional supplement.

19. The nutritional supplement of claim 14, wherein the potassium compounds comprise one or more of potassium citrate, potassium bicarbonate and potassium chloride.

20. The nutritional supplement of claim 14, wherein the potassium compounds comprise potassium citrate, potassium bicarbonate and potassium chloride.
